(19) United States
(12) Patent Application Publication
(10) Pub. No.: US 2024/0217918 A1
(43) Pub. Date: Jul. 4, 2024

(54) SOLID FORMS OF (R)-OXYBUTYNIN D-MALATE

(71) Applicant: APNIMED, INC. (DELAWARE), Cambridge, MA (US)

(72) Inventors: Sean JOHNSTON, Doylestown, PA (US); Dennis MOLNAR, Hopkinton, NH (US)

(21) Appl. No.: 18/289,126

(22) PCT Filed: May 4, 2022
(86) PCT No.: PCT/US2022/027573

§ 371 Date: Nov. 1, 2023

Related U.S. Application Data
(63) Continuation-in-part of application No. PCT/US2021/030571, filed on May 4, 2021.
(60) Provisional application No. 63/248,684, filed on Sep. 27, 2021.

(51) Int. Cl. C07C 219/20; A61K 31/222; C07C 51/43; C07C 59/245; C07C 59/255; C07C 221/00

(52) U.S. Cl. CPC C07C 219/20; A61K 31/222; C07C 51/43; C07C 59/245; C07C 59/255; C07C 221/00; C07B 2200/13

(57) ABSTRACT

(R)-Oxybutynin D-malate salts, including crystalline and amorphous forms, are prepared and characterized. Uses of (R)-oxybutynin D-malate for obstructive sleep apnea (OSA) treatment are also disclosed. Solid forms of (R)-oxybutynin L-tartrate are also disclosed.

### CROSS-REFERENCE TO RELATED APPLICATIONS

This application claims priority to U.S. provisional application 63/248,684, filed Sep. 27, 2021 and PCT application no. PCT/US2021/030571, filed May 4, 2021, the entire contents of each of which are incorporated herein by reference.

### TECHNICAL FIELD

The present invention discloses solid forms of (R)-oxybutynin D-malate, along with pharmaceutical compositions thereof, preparation methods thereof, and uses thereof. Solid forms of (R)-oxybutynin L-tartrate are also disclosed.

### BACKGROUND

Oxybutynin and its derivatives are typically taken by mouth or applied to the skin and are applicable as bronchodilators or a remedy for overactive bladder. In addition, oxybutynin exerts a direct antispasmodic effect on various forms of smooth muscle, mainly by inhibiting the action of acetylcholine on smooth muscle as an anti-cholinergic drug and the like. Racemic oxybutynin is marketed in the hydrochloride form. The chemical name for oxybutynin is 4-(diethylamino)but-2-yn-1-yl 2-cyclohexyl-2-hydroxy-2-phenylacetate for which the chemical structure is provided below as I:

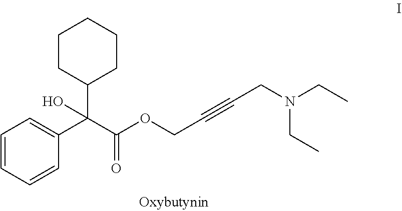 [SMILES: CCN(CC)CC#CCOC(=O)C(O)(c1ccccc1)C1CCCCC1]

The R enantiomer of oxybutynin has utility as an active pharmaceutical ingredient for treatment of conditions associated with pharyngeal airway collapse, such as obstructive sleep apnea. See WO 2019/152475 A1. Thus, there is a need for pharmaceutically useful forms of oxybutynin, including (R)-oxybutynin.

### BRIEF DESCRIPTION OF THE DRAWINGS

The following figures are provided by way of example and are not intended to limit the scope of the claimed invention.

FIG. 1 is an XRPD pattern for (R)-oxybutynin D-malate Form A.

FIG. 2 is a FT-Raman spectrum for (R)-oxybutynin D-malate Form A.

FIG. 3 shows DSC and TGA traces for (R)-oxybutynin D-malate Form A.

FIG. 4 shows the structure of (R)-oxybutynin D-malate Form A as analyzed by SCXRD.

FIG. 5A and FIG. 5B show the hydrogen bonding scheme and packing of crystals for (R)-oxybutynin D-malate Form A as analyzed by SCXRD.

FIG. 6 is an XRPD pattern for (R)-oxybutynin L-tartrate.

FIG. 7 is a FT-Raman spectrum for (R)-oxybutynin L-tartrate.

FIG. 8 shows DSC and TGA traces for (R)-oxybutynin L-tartrate.

### DETAILED DESCRIPTION

### I. (R)-Oxybutynin D-Malate

The present invention relates, in part, to novel solid forms of (R)-oxybutynin, e.g., novel salts and novel crystalline forms. Generally, a solid compound's efficacy as a drug can be affected by the properties of the solid it comprises. As set forth in the Example section below, a crystalline form of (R)-oxybutynin D-malate was prepared and characterized. The (R) enantiomer of oxybutynin D-malate is provided below as II:

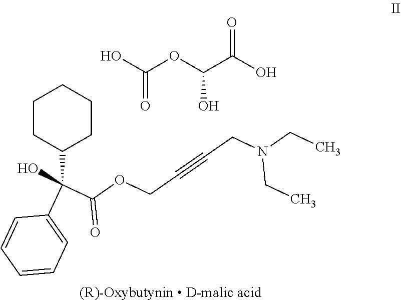

An amorphous form of (R)-oxybutynin D-malate was also prepared.

The definitions provided herein are meant to clarify, but not limit, the terms defined. If a term used herein is not specifically defined, such term should not be considered indefinite. Rather, terms are used within their accepted meanings.

As used herein, (R)-oxybutynin D-malate refers to a D-malic acid salt form wherein the molar ratio of (R)-oxybutynin and D-malic acid is approximately 1, e.g., from about 0.75 to about 1.25, from about 0.9 to about 1.1, from about 1.0 to about 1.25, or from 0.75 to about 1.0. Small changes in the amount of assayed D-malic acid can be attributed to, without limitation, measurement variability and the presence of an excess of either reagent through processing and/or isolation.

As used herein, “crystalline” refers to a solid having a highly regular chemical structure. In particular, a crystalline free base or salt form may be produced as one or more single crystalline forms.

The term “substantially crystalline” refers to forms that may be at least a particular weight percent crystalline. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, substantially crystalline refers to a free base or salt form that is at least 70% crystalline. In other embodiments, substantially crystalline refers to a free base or salt form that is at least 90% crystalline.

As used herein, “amorphous” refers to a solid material comprising non-crystalline materials. In certain embodiments, an amorphous sample of a material may be prepared by lyophilization of a mixture of the material with a solvent, wherein the mixture may be homogeneous (e.g., solution) or heterogeneous (e.g., a slurry).

The term “substantially free” refers to forms and compositions that may be at least a particular weight percent free of impurities and/or crystalline compound. Particular weight percentages are 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 60% and 100% free of impurities and/or crystalline compound. In some embodiments, substantially free refers to a free base or salt form that is at least 70% pure. In other embodiments, substantially free refers to a free base or salt form that is at least 90% pure. In other embodiments, substantially free of crystalline compound refers to a composition having less than about 30%, less than about 20%, less than about 15%, less than about 10%, less than about 5%, less than about 1% of crystalline compound.

In many embodiments disclosed herein, (R)-oxybutynin D-malate is disclosed as having a crystalline structure.

In certain embodiments, crystalline structures in this disclosure can be identified by having one or more characteristics peaks in an XRPD spectrum, as disclosed herein.

In some embodiments, crystalline structures in this disclosure have one or more characteristics endothermic peaks in differential scanning calorimetry, as disclosed herein.

In certain embodiments, methods of preparing a crystalline form of (R)-oxybutynin D-malate are provided. Further embodiments describe the conversion to, and preservation of a crystalline form of (R)-oxybutynin D-malate that has desired stability under expected storage conditions.

Certain embodiments disclosed herein provide crystalline (R)-oxybutynin D-malate.

Certain embodiments disclosed herein provide crystalline (R)-oxybutynin D-malate of Form A.

In some embodiments, the crystalline (R)-oxybutynin D-malate (e.g., Form A) has an X-ray Powder Diffraction (XRPD) pattern comprising at least three peaks selected from the group consisting of peaks at 11.0, 14.3, 17.5, 19.8 and 22.0° 2θ=0.2° 2θ.

In some embodiments, the crystalline (R)-oxybutynin D-malate (e.g., Form A) has an XRPD pattern comprising peaks at 11.0, 17.5 and 22.0° 2θ+0.2° 2θ.

In some embodiments, the crystalline (R)-oxybutynin D-malate (e.g., Form A) has an XRPD pattern comprising peaks at 11.0, 14.3, 17.5, 19.8 and 22.0° 2θ+0.2° 2θ.

In some embodiments, the crystalline (R)-oxybutynin D-malate (e.g., Form A) has an XRPD pattern comprising at least five, six, seven, eight, or nine peaks selected from the group consisting of peaks at 11.0, 14.3, 17.5, 18.8, 19.8, 21.4, 22.0, 23.3, 24.1 and 33.2° 2θ+0.2° 2θ.

In some embodiments, the crystalline (R)-oxybutynin D-malate (e.g., Form A) has an XRPD pattern comprising peaks at 11.0, 14.3, 17.5, 18.8, 19.8, 21.4, 22.0, 23.3, 24.1 and 33.2° 2θ+0.2° 2θ.

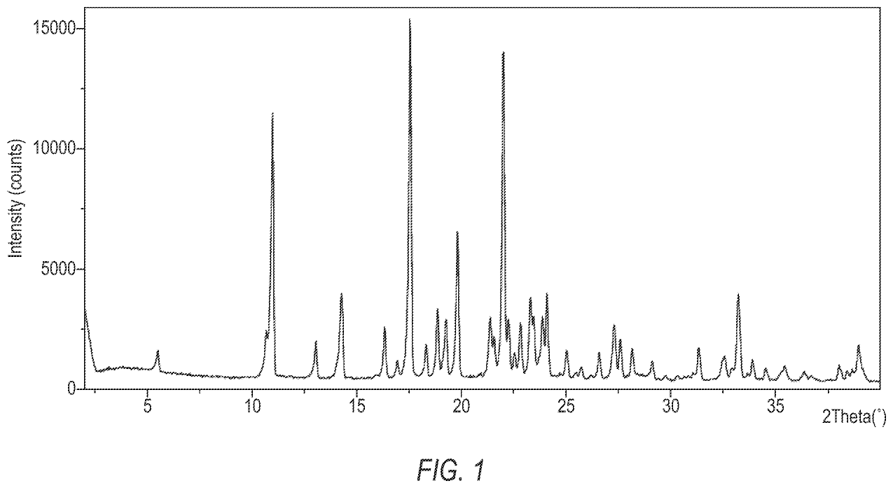

In some embodiments, the crystalline (R)-oxybutynin D-malate (e.g., Form A) has an XRPD pattern substantially as shown in FIG. 1.

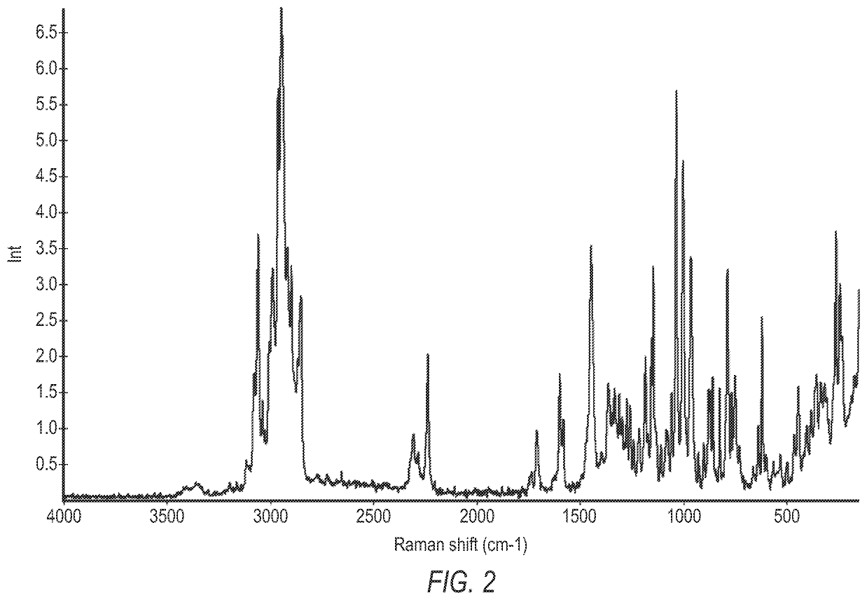

Certain embodiments disclosed herein provide crystalline (R)-oxybutynin D-malate (e.g., Form A), having an FT-Raman spectrum substantially as shown in FIG. 2.

Certain embodiments disclosed herein provide crystalline (R)-oxybutynin D-malate (e.g., Form A), having a differential scanning calorimetry (DSC) thermogram displaying a melting onset at about 108.1° C. and/or an endothermic peak at about 109.4° C.

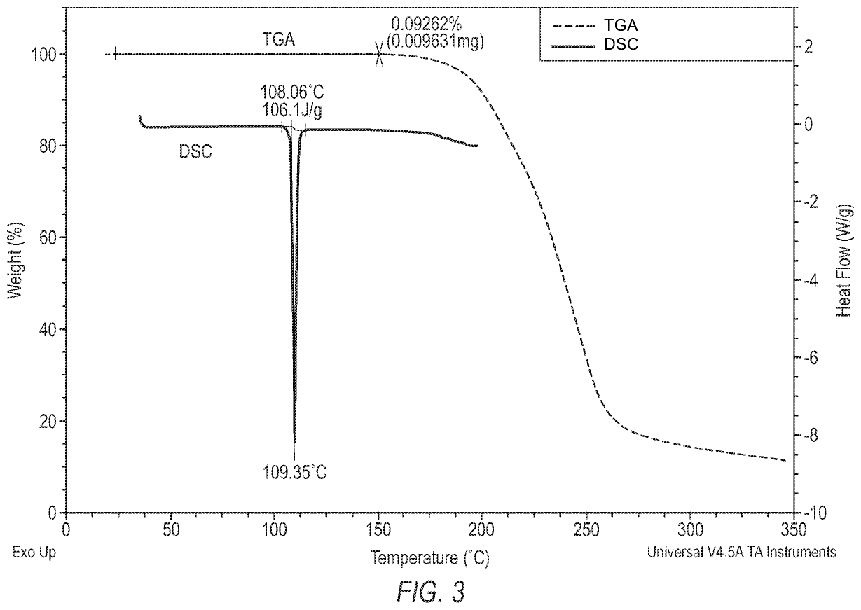

Certain embodiments disclosed herein provide crystalline (R)-oxybutynin D-malate (e.g., Form A), having a differential scanning calorimetry (DSC) thermogram substantially as shown in FIG. 3.

Certain embodiments disclosed herein provide crystalline (R)-oxybutynin D-malate (e.g., Form A), having the following unit cell parameters:

| a[Å] | b[Å] | c[Å] | α[°] | β[°] | γ[°] | V[Å³] |
|---|---|---|---|---|---|---|
| 8.5 | 9.7 | 16.6 | 90 | 102.7 | 90 | 1338 |

Certain embodiments disclosed herein provide a composition comprising (R)-oxybutynin wherein at least 5% w/w, at least 10% w/w, at least 25% w/w, at least 50% w/w, at least 75% w/w, at least 80% w/w, at least 90% w/w, at least 95% w/w, at least 98% w/w, at least 99% w/w, or at least 99.9% w/w of the total amount of (R)-oxybutynin is (R)-oxybutynin D-malate Form A.

Certain embodiments disclosed herein provide a pharmaceutical composition comprising (R)-oxybutynin D-malate Form A in any of its specified embodiments and one or more pharmaceutically acceptable excipients.

Certain embodiments disclosed herein provide amorphous (R)-oxybutynin D-malate.

Certain embodiments disclosed herein provide one or more crystalline and/or amorphous forms of (R)-oxybutynin D-malate dispersed into a matrix.

Certain embodiments are disclosed comprising a dosage form of (R)-oxybutynin D-malate comprising from about 0.1 to about 25 mg, from about 0.1 to about 15 mg, from about 0.1 to about 10 mg, from about 1 to about 25 mg, from about 1 to about 20 mg, from about 1 to about 15 mg, from about 1 to about 10 mg, from about 1 to about 5 mg, from about 2 to about 25 mg, from about 2 to about 20 mg, from about 2 to about 15 mg, from about 2 to about 10 mg, from about 2 to about 5 mg, from about 5 to about 25 mg, from about 5 to about 20 mg, from about 5 to about 15 mg, or from about 5 to about 10 mg of (R)-oxybutynin D-malate in one or more crystalline and/or amorphous forms, optionally wherein said one or more crystalline and/or amorphous forms are dispersed in a solid or liquid matrix.

### (R)-Oxybutynin L-Tartrate

Also disclosed herein are solid forms of (R)-oxybutynin L-tartrate. In some embodiments, the (R)-oxybutynin L-tartrate is a crystalline solid.

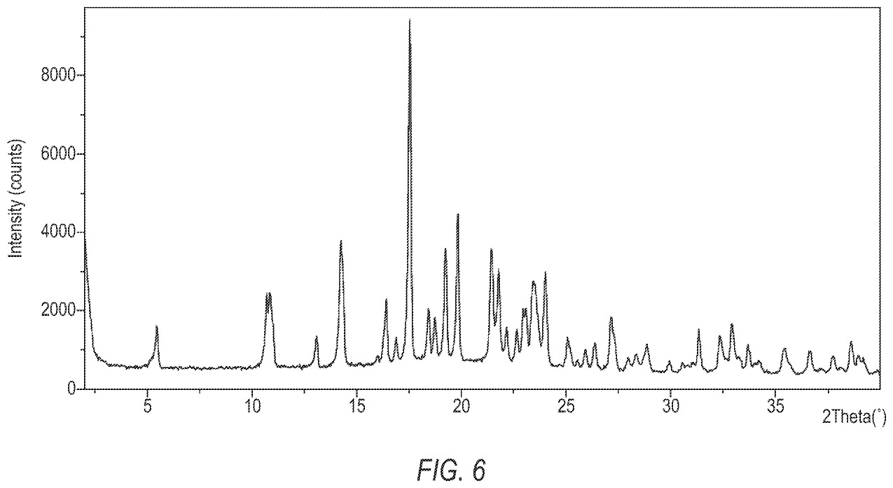

In some embodiments, the crystalline (R)-oxybutynin L-tartrate has an XRPD pattern substantially as shown in FIG. 6.

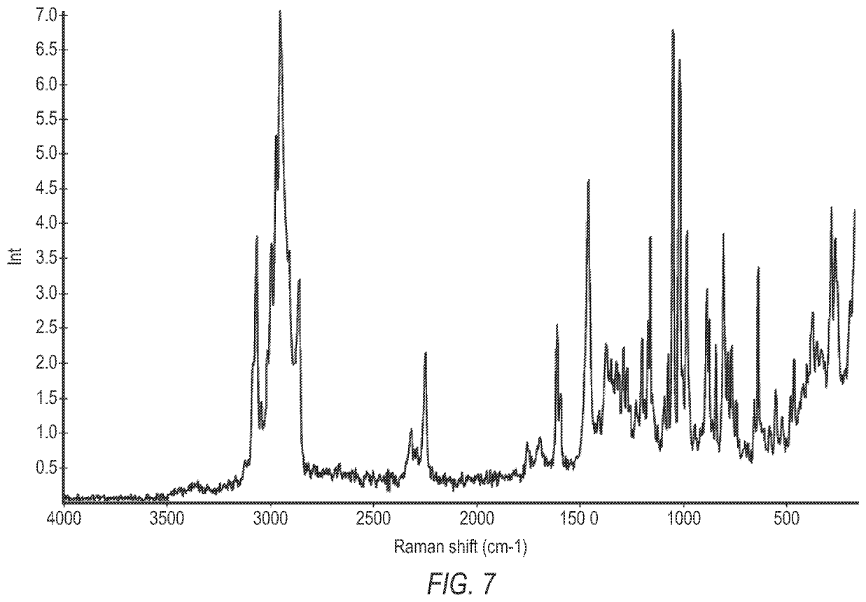

In some embodiments, the crystalline (R)-oxybutynin L-tartrate has an FT-Raman spectrum substantially as shown in FIG. 7.

In some embodiments, the crystalline (R)-oxybutynin L-tartrate has a differential scanning calorimetry (DSC) thermogram displaying an endotherm (e.g., large endotherm) at about 92° C.

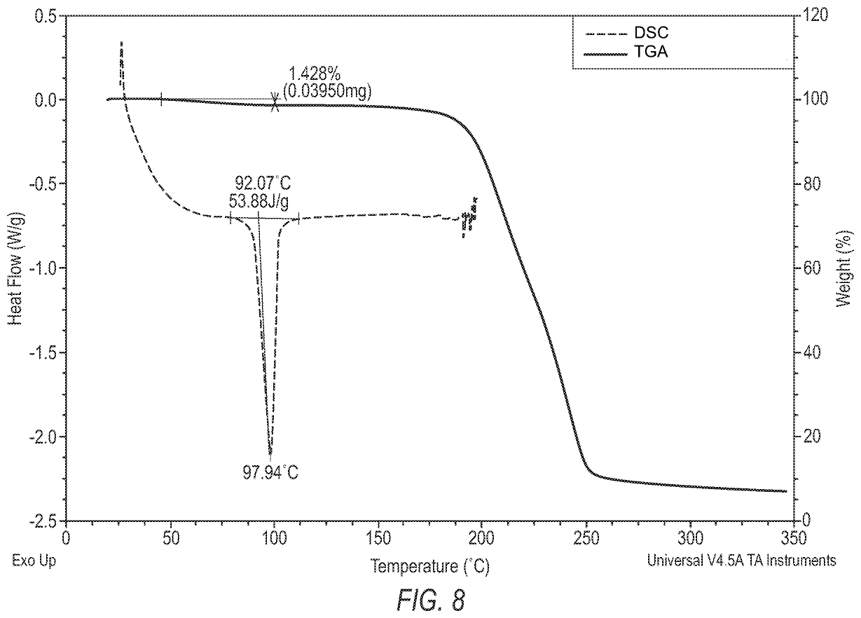

In some embodiments, the crystalline (R)-oxybutynin L-tartrate has a DSC thermogram substantially as shown in FIG. 8.

### II. Pharmaceutical Compositions of (R)-Oxybutynin D-Malate

Provided herein are pharmaceutical compositions comprising one or more forms of (R)-oxybutynin D-malate, and a physiologically acceptable carrier (also referred to as a pharmaceutically acceptable carrier or solution or diluent). Such carriers and solutions include pharmaceutically acceptable salts and solvates of compounds used in the methods of the instant invention, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington's Pharmaceutical Sciences, 17th edition, ed. Alfonso R. Gennaro, Mack Publishing Company, Eaton, Pa. (1985), which is incorporated herein by reference.

The term “pharmaceutically acceptable carrier” refers to a carrier that does not cause an allergic reaction or other untoward effect in a subject to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

The one or more crystalline and/or amorphous forms of (R)-oxybutynin D-malate disclosed herein and pharmaceutical compositions thereof may be formulated into unit dosage forms, meaning physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. In certain embodiments, the compounds may be formulated for controlled release.

The one or more crystalline and/or amorphous forms of (R)-oxybutynin D-malate disclosed herein and pharmaceutical compositions thereof may be formulated according to any available conventional method. In the formulation, generally used additives such as a diluent, a binder, a disintegrant, a lubricant, a colorant, a flavoring agent, and if necessary, a stabilizer, an emulsifier, an absorption enhancer, a surfactant, a pH adjuster, an antiseptic, an antioxidant and the like can be used. For the purpose of oral therapeutic administration, the active compound(s) can be incorporated with excipients and used in the form of pills, tablets, troches, or capsules, e.g., gelatin capsules. Oral compositions can also be prepared using a fluid carrier. Pharmaceutically compatible binding agents, and/or adjuvant materials can be included as part of the composition. Dosage forms including a tablet, a powder, a subtle granule, a granule, a coated tablet, a capsule, a syrup, a troche, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, crospovidone or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

Some embodiments disclosed herein provide a pharmaceutical dosage form comprising (R)-oxybutynin D-malate Form A in an amount of about 0.1 mg, about 0.5 mg, about 0.75 mg, about 1 mg, about 1.5 mg, about 2 mg, about 2.5 mg, about 3 mg, about 4 mg, about 5 mg, about 7.5 mg, about 10 mg, about 12.5 mg, about 15 mg, about 17.5 mg, about 20 mg, about 22.5 mg, or about 25 mg. In certain embodiments, at least 80%, at least 85%, at least 90%, at least 95%, at least 98%, or at least 99.5% of the (R)-oxybutynin in the pharmaceutical dosage form is (R)-oxybutynin D-malate Form A.

Certain embodiments disclosed herein provide a drug dosage form as a tablet comprising about 0.1 mg, about 0.5 mg, about 0.75 mg, about 1 mg, about 1.5 mg, about 2 mg, about 2.5 mg, about 3 mg, about 4 mg, about 5 mg, about 7.5 mg, about 10 mg, about 12.5 mg, about 15 mg, about 17.5 mg, about 20 mg, about 22.5 mg, or about 25 mg of (R)-oxybutynin D-malate Form A. In certain embodiments, at least 80%, at least 85%, at least 90%, at least 95%, at least 98%, or at least 99.5% of the (R)-oxybutynin in the tablet is (R)-oxybutynin D-malate Form A.

Certain embodiments disclosed herein provide a pharmaceutical composition comprising about 0.1 mg, about 0.5 mg, about 0.75 mg, about 1 mg, about 1.5 mg, about 2 mg, about 2.5 mg, about 3 mg, about 4 mg, about 5 mg, about 7.5 mg, about 10 mg, about 12.5 mg, about 15 mg, about 17.5 mg, about 20 mg, about 22.5 mg, or about 25 mg of (R)-oxybutynin D-malate Form A, and one or more pharmaceutically acceptable excipients. In certain embodiments, at least 80%, at least 85%, at least 90%, at least 95%, at least 98%, or at least 99.5% of the (R)-oxybutynin in the pharmaceutical composition is (R)-oxybutynin D-malate Form A.

Certain embodiments disclosed herein comprise (R)-oxybutynin D-malate Form A or pharmaceutical compositions thereof substantially free of other crystalline or amorphous forms. For example, in some embodiments, the (R)-oxybutynin D-malate Form A or pharmaceutical composition thereof comprises 90%, 91%, 92%, 93%, 94%, 95%, 96%, 97%, 98%, 99%, 99.5%, 99.9% by weight of Form A relative to other crystalline or amorphous forms of (R)-oxybutynin D-malate.

### III. Use of (R)-Oxybutynin D-Malate

Provided herein in some embodiments are methods of treating a subject having a condition associated with pharyngeal airway collapse, the method comprising administering to a subject in need thereof an effective amount of (i) a norepinephrine reuptake inhibitor (NRI) and (ii) (R)-oxybutynin D-malate. Also provided herein is the use of (i) a norepinephrine reuptake inhibitor (NRI) and (ii) (R)-oxybutynin D-malate in the manufacture of a medicament for treating a condition associated with pharyngeal airway collapse. Also provided herein is (i) A norepinephrine reuptake inhibitor (NRI) and (ii) (R)-oxybutynin D-malate, or a pharmaceutical composition thereof, for use in treating a condition associated with pharyngeal airway collapse.

In some embodiments, the (R)-oxybutynin D-malate is crystalline. In some embodiments, the (R)-oxybutynin D-malate is crystalline Form A. In some embodiments, the method comprises administering a pharmaceutical composition comprising an NRI and (R)-oxybutynin D-malate (e.g., Form A). In some embodiments, the condition is sleep apnea, e.g., obstructive sleep apnea. In some embodiments, the condition is snoring, e.g., simple snoring. In some embodiments, the NRI is atomoxetine or a pharmaceutically acceptable salt thereof. In some embodiments, the NRI is reboxetine or a pharmaceutically acceptable salt thereof.

Provided herein in some embodiments are methods of treating a subject having a condition associated with pharyngeal airway collapse, the method comprising administering to a subject in need thereof an effective amount of (i) atomoxetine or a pharmaceutically acceptable salt thereof; and (ii) (R)-oxybutynin D-malate. In some embodiments, the methods comprise administering to the subject a therapeutically effective amount of crystalline (R)-oxybutynin D-malate (e.g., Form A), or a pharmaceutical composition thereof.

In some embodiments, provided herein are methods of treating a subject having a condition associated with pharyngeal airway collapse further comprising administering to a subject in need thereof a hypnotic.

In some embodiments, provided herein are methods of treating a subject having a condition associated with pharyngeal airway collapse further comprising administering to a subject in need thereof a carbonic anhydrase inhibitor.

Exemplary norepinephrine reuptake inhibitors (NRIs) include the selective NRIs Amedalin (UK-3540-1), Atomoxetine (Strattera), CP-39,332, Daledalin (UK-3557-15), Edivoxetine (LY-2216684), Esreboxetine, Lortalamine (LM-1404), Nisoxetine (LY-94,939), Reboxetine (Edronax, Vestra), Talopram (Lu 3-010), Talsupram (Lu 5-005), Tandamine (AY-23,946), Viloxazine (Vivalan); non-selective NRIs include Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine (GW-320,659), Maprotiline, Methylphenidate, Milnacipran, Nefazodone, Nortriptyline, Phendimetrazine, Phenmetrazine, Protryptyline, Radafaxine (GW-353,162), Tapentadol (Nucynta), Teniloxazine (Lucelan, Metatone) and Venlafaxine and pharmaceutically acceptable salts thereof.

In some embodiments, the norepinephrine reuptake inhibitor is atomoxetine or a pharmaceutically acceptable salt thereof. In other embodiments, the norepinephrine reuptake inhibitor is reboxetine or a pharmaceutically acceptable salt thereof. In still other embodiments, the norepinephrine reuptake inhibitor is a combination of atomoxetine and reboxetine or pharmaceutically acceptable salts thereof.

Oxybutynin is an antimuscarinic drug and a muscarinic receptor antagonist and refers to the racemic mixture of (R) and (S) enantiomers. (R)-oxybutynin refers to the (R) enantiomer. In compositions described herein, the (R)-oxybutynin is in an enantiomeric excess of (R)-oxybutynin relative to its enantiomeric pair (i.e., (S)-oxybutynin). The enantiomeric excess of (R)-oxybutynin in these compositions may be ≥80%, ≥90%, ≥95%, ≥98%, ≥99%, ≥99.5%, ≥99.8% or ≥99.9%.

The carbonic anhydrase inhibitor may be selected from the group consisting of acetazolamide, dichlorophenamide, dorzolamide, brinzolamide, methazolamide, zonisamide, ethoxzolamide, topiramate, sultiame, and any combinations thereof or pharmaceutically acceptable salts thereof. In some embodiments, the carbonic anhydrase inhibitor is acetazolamide or a pharmaceutically acceptable salt thereof.

In some embodiments, hypnotics may be incorporated into the compositions, e.g., zolpidem, zopiclone, eszopiclone, trazodone, zaleplon, benzodiazepines, gabapentin, tiagabine, and xyrem or pharmaceutically acceptable salts thereof.

In some embodiments, a patient is a human subject.

In some embodiments, the methods include administering a dose of from about 20 mg to about 150 mg atomoxetine or a pharmaceutically acceptable salt thereof, from about 20 mg to about 100 mg atomoxetine or a pharmaceutically acceptable salt thereof, from about 50 mg to about 100 mg atomoxetine or a pharmaceutically acceptable salt thereof, or from about 75 mg to about 100 mg atomoxetine or a pharmaceutically acceptable salt thereof. In some embodiments, the methods include administering a dose of from about 0.1 mg to about 25 mg (R)-oxybutynin D-malate, from about 1 mg to about 20 mg (R)-oxybutynin D-malate, from about 1 mg to about 10 mg (R)-oxybutynin D-malate, from about 1 mg to about 5 mg (R)-oxybutynin D-malate, or from about 2.5 mg to about 7.5 mg (R)-oxybutynin D-malate. In other embodiments, the methods include administering a dose of from about 20 mg to about 150 mg atomoxetine or a pharmaceutically acceptable salt thereof in combination with about 0.1 mg to about 25 mg (R)-oxybutynin D-malate, from about 20 mg to about 150 mg atomoxetine or a pharmaceutically acceptable salt thereof in combination with about 1 mg to about 20 mg (R)-oxybutynin D-malate, from about 20 mg to about 150 mg atomoxetine or a pharmaceutically acceptable salt thereof in combination with about 1 mg to about 10 mg (R)-oxybutynin D-malate, from about 20 mg to about 150 mg atomoxetine or a pharmaceutically acceptable salt thereof in combination with about 1 mg to about 5 mg (R)-oxybutynin D-malate, or from about 20 mg to about 150 mg atomoxetine or a pharmaceutically acceptable salt thereof in combination with about 2.5 mg to about 7.5 mg (R)-oxybutynin D-malate.

An effective amount can be administered in one or more administrations, applications or dosages. The compositions can be administered from one or more times per day to one or more times per week; including once every other day. In some embodiments, the compositions are administered daily, e.g., before bed time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

### IV. Preparation and Characterization of Crystalline (R)-Oxybutynin D-Malate Form A

Provided herein are methods for preparing (R)-oxybutynin D-malate Form A. In some embodiments, crystalline (R)-oxybutynin D-malate Form A can be prepared by combining (R)-oxybutynin free base with D-malic acid (e.g., about 1.0 eq) in solvent with ethyl acetate and methyl tert-butyl ether (MTBE) (e.g., in a volume ratio of 1:4 of ethyl acetate:MTBE). Crystals can then be isolated from the resulting mixture.

In some embodiments, crystalline (R)-oxybutynin D-malate Form A can be prepared by adding D-malic acid to racemic oxybutynin in the presence of a solvent. In some embodiments, the solvent is 2-propanol. In some embodiments, seed crystals of (R)-oxybutynin D-malate Form A are utilized.

FIG. 1 provides the XRPD pattern for (R)-oxybutynin D-malate Form A, and the corresponding peaks are provided in Table 1 below indicating that the (R)-oxybutynin D-malate Form A material is composed primarily or exclusively of a single crystalline phase. (R)-oxybutynin D-malate Form A is a mono D-malic acid salt of (R)-oxybutynin. Form A is a non-solvated crystal. It was produced as a white crystalline powder with sharp endotherm at about 108° C. and negligible weight loss up to 150° C. as measured by TGA.

### EXAMPLES

### Instrument and Methodology

### A. X-Ray Powder Diffraction (XRPD)

XRPD diffractograms were acquired on PANalytical X'Pert Pro diffractometer using Ni-filtered Cu Ka (45 kV/40 mA) radiation and a step size of 0.03° 2θ and X'celerator™ RTMS (Real Time Multi-Strip) detector. Configuration on the incidental beam side: variable divergence slits (10 mm irradiated length), 0.04 rad Soller slits, fixed anti-scatter slit (0.50°), and 10 mm beam mask. Configuration on the diffracted beam side: variable anti-scatter slit (10 mm observed length) and 0.04 rad Soller slits. Samples were mounted flat on zero-background Si wafers.

### B. FT-Raman Spectroscopy

Raman spectra were collected with a Nicolet NXR9650 (Thermo Scientific) equipped with 1064 nm Nd:YVO₄ excitation laser, InGaAs and liquid-N₂ cooled Ge detectors and a MicroStage. All spectra were acquired at 4 cm-1 resolution using Happ-Genzel apodization function and 2-level zero-filling.

### C. Differential Scanning Calorimetry (DSC)

DSC was conducted with a TA Instruments Q200 or Q2000 differential scanning calorimeter equipped with an autosampler and a refrigerated cooling system under 40 mL/min or 50 mL/min N₂ purge for the Q2000 and Q200, respectively. DSC thermograms of samples were obtained at 10° C./min in crimped Al pans. The temperatures of exothermic and endothermic transitions recorded via DSC analysis are reported as onset values.

### D. Thermogravimetric Analysis (TGA)

TGA thermograms were obtained with a TA Instruments Q500 thermogravimetric analyzer under 40 mL/min N₂ purge for balance and 60 mL/min for sample in Al pans. TGA thermograms of samples were obtained at 10° C./min.

### E. Gravimetric Vapor Sorption (GVS)

GVS experiments were conducted on a Surface Measurement Systems DVS-Advantage. The experiments were performed at 25° C. The instrument was operated in step mode and the relative humidity was increased in 10% RH increments from 0% RH to 90% RH with an extra step at 75% RH, then decreased from 90% RH to 0% RH with an extra step at 75% RH. The mass equilibrium criterion was set at 0.005% change in mass over time (dm/dt). A minimum step time of 15 minutes and a maximum step time of 240 minutes were specified.

### Example 1: Production of Crystalline (R)-Oxybutynin D-Malate and Seed Crystals

(R)-oxybutynin free-base was prepared from (R)-oxybutynin HCl salt as follows. (R)-oxybutynin HCl salt (506 mg, 1.28 mol) was dissolved in 3.0 mL of water (6 vol) at RT. One equivalent of aqueous 1 M NaOH was added (1.28 mL), resulting in a gum. The aqueous layer was decanted from the gum and any free-base was extracted with hexane. The gum was dissolved in hexane and washed with water. The combined hexane layers were concentrated in vacuo, yielding 452 mg of (R)-oxybutynin free base as an oil (98% yield).

Approximately 20 mg of (R)-oxybutynin free-base was combined with 1.0 eq of D-malic acid. A 1:4 ratio (by volume) of ethyl acetate/MTBE was added. Crystals were produced and isolated. Thus, crystalline (R)-oxybutynin D-malate was produced, which crystals were used as seed crystals for further experiments.

### Example 2: Production and Characterization of (R)-Oxybutynin D-Malate Form A

(R)-oxybutynin D-malate salt was prepared and its solid forms were analyzed and characterized. A supplied lot of (R)-oxybutynin free-base was used as a starting material. The (R)-oxybutynin free base (9.52 g) was dissolved in 2-propanol (76 mL) at 50° C. One equivalent of D-malic acid (3.69 g) was added, and the mixture stirred at 50° C. for 5 minutes until dissolution was observed. The solution was cooled to 40° C. and seeded with (R)-oxybutynin D-malate crystals. After 10 min, the heat was turned off and the mixture cooled to RT with stirring for 20 h. The solids were isolated by vacuum filtration and air dried for 30 min. The yield was 83% (10.9 g, 22.1 mmol) of (R)-oxybutynin D-malate salt as white powder. Form A is a non-solvated crystal form.

The solid-state attributes of the prepared batch were determined by the following analytical techniques: X-ray Powder Diffraction (XRPD), FT-Raman Spectroscopy, Differential Scanning calorimetry (DSC), and Thermogravimetric Analysis (TGA).

The XRPD pattern of the (R)-oxybutynin D-malate salt is shown in FIG. 1, and was a crystalline material designated as Form A. The relative XRPD peak intensities are shown below in Table 1.

TABLE 1

| | | | Rel. Int. |
|---|---|---|---|
| Pos. [°2Th.] | Height [cts] | d-spacing [Å] | [%] |
| 5.5 | 857.4 | 16.1 | 5.8 |
| 11.0 | 10478.9 | 8.1 | 70.4 |
| 13.0 | 1531.8 | 6.8 | 10.3 |
| 14.3 | 3487.1 | 6.2 | 23.4 |
| 16.3 | 1978.2 | 5.4 | 13.3 |
| 17.5 | 14886.5 | 5.1 | 100.0 |
| 18.3 | 1391.1 | 4.8 | 9.3 |
| 18.8 | 2728.6 | 4.7 | 18.3 |
| 19.3 | 2270.2 | 4.6 | 15.3 |
| 19.8 | 5809.2 | 4.5 | 39.0 |
| 21.4 | 2309.7 | 4.2 | 15.5 |
| 22.0 | 13412.2 | 4.0 | 90.1 |
| 22.8 | 2087.8 | 3.9 | 14.0 |
| 23.3 | 3095.7 | 3.8 | 20.8 |
| 24.1 | 3154.7 | 3.7 | 21.2 |
| 27.3 | 2096.9 | 3.3 | 14.1 |
| 28.1 | 1229.8 | 3.2 | 8.3 |
| 31.3 | 1834.5 | 2.9 | 12.3 |
| 33.2 | 3518.2 | 2.7 | 23.6 |
| 38.9 | 1459.4 | 2.3 | 9.8 |

FT-Raman spectrum is shown in FIG. 2.

DSC and TGA analyses are shown in FIG. 3. DSC analysis showed a sharp endotherm at 108° C. (ΔH=106 J/g). TGA analysis showed a negligible (<0.1% wt loss) up to 150° C. with further weight loss above 150° C., likely due to decomposition. GVS analysis showed 0.1% moisture uptake between 0-90% RH, demonstrating that Form A is a non-hygroscopic solid. No change in crystal form was observed after the GVS test. Proton NMR confirmed a mono-salt (actual ration of 1.0:1 counterion: API ratio)

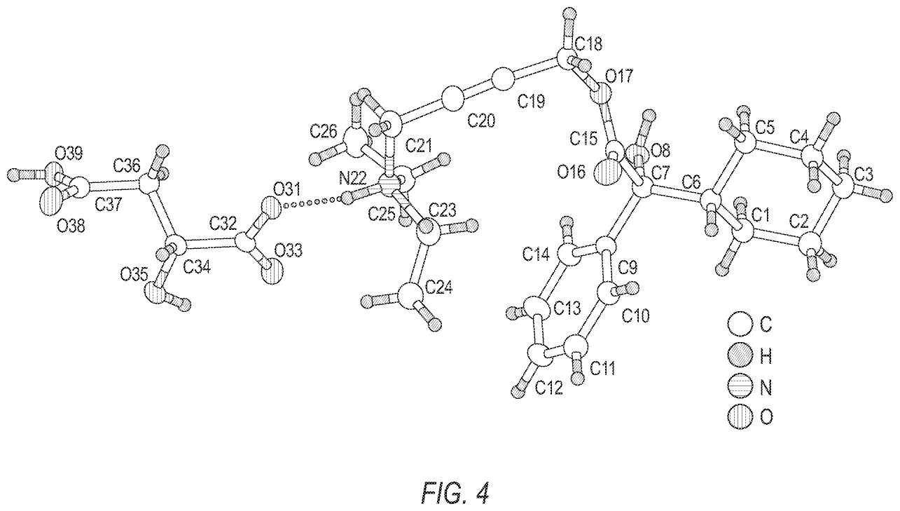

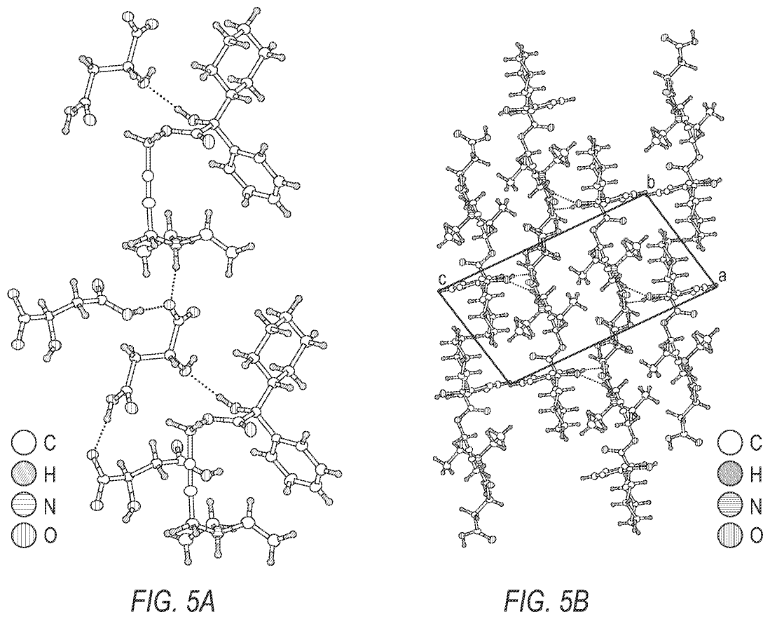

Large crystals of Form A, suitable for single-crystal X-ray diffraction (SCXRD) were observed from a visual solubility experiment in ethyl acetate and were submitted for SCXRD analysis. The SCXRD data indicated the atomic connectivity is consistent with the proposed molecular structure, and the absolute configuration of both oxybutynin and malic acid are R. The calculated powder pattern was consistent with the XRPD pattern of FIG. 1. A Bruker D8 Venture Photon II CPAD diffractometer equipped with CuKα INCOATEC ImuS micro-focus source (λ=1.54178 Å) was used for SCXRD. Temperature was 100K. The structure as analyzed by SCXRD is shown in FIG. 4. The molecule showed a monoclinic P2₁ structure that was non-centrosymmetric (chiral). The absolute configuration of both chiral C7 and C34 atoms was R. Packing of crystals is shown in FIG. 5 including (A) hydrogen bonding scheme and (B) packing as viewed along ˜[010] or b direction. Unit cell parameters were determined at room temperature using Difference Vectors method based on 64 reflections harvested from 46, 0.5° diffraction frames. The parameters were refined during data integration and are based on 62 reflections recorded between 8 and 1.02 Å resolution. Unit cell parameters are provided below.

| a[Å] | b[Å] | c[Å] | α[°] | β[°] | γ[°] | V[Å³] |
|---|---|---|---|---|---|---|
| 8.529(6) | 9.70(1) | 16.59(1) | 90 | 102.73(3) | 90 | 1338(2) |

### Example 3: Solubility Assessment of Form A

Solubility of Form A was visually assessed in 12 diverse solvents at RT and 40° C. The solubility data are shown in Table 2. The solubility was visually estimated by dosing small aliquots of solvent into a fixed amount of a solid (˜10 mg) until the dissolution point or a maximum volume (1.8 mL) was reached. Samples that contained undissolved solids at RT were heated to 40° C. for 1 h and the dissolution was assessed visually.

TABLE 2

| | | Solubility at RT | Solubility at 40 ° C. |
|---|---|---|---|
| # | Solvent (v/v) | (mg/mL) | (mg/mL) |
| 1 | Dimethyl sulfoxide (DMSO) | >400 | n/a |
| 2 | Methanol (MeOH) | >400 | n/a |
| 3 | Water | 100-400 | n/a |
| 4 | 2-propanaol:water (9:1) | 100-400 | n/a |
| 5 | Tetrahydrofuran (THF) | 100-400 | n/a |
| 6 | Dichloromethane (DCM) | 100-400 | n/a |
| 7 | Acetonitrile (MeCN) | 20-50 | n/a |
| 8 | 2-Propanol (IPA) | 10-20 | n/a |
| 9 | Ethyl acetate (EtOAc) | <6 | >6 |
| 10 | Methyl t-butyl ether (MTBE) | <6 | >6 |
| 11 | Toluene | <6 | <6 |
| 12 | Isopropyl ether (IPE) | <6 | <6 |

### Example 4: Crystal Form Screen

A crystal form screen was comprised of ˜144 crystallization experiments and involved 48 solvents, three crystallization modes (slurry ripening, cooling, evaporation), and a temperature range of 5-40° C. Form A was utilized as the input form. Products were obtained from thermocycling (TC), cooling (RC), and evaporation (EV) experiments.

The crystallization modes were as follows: TC: Stirring API suspensions while cycling the temperature between 40-5° C. for 2 d; RC: Cooling clarified API solutions from 40° C. to 4° C., followed by a hold at 4° C. for 6 d; EV: Slow evaporation of solvents from API solutions at RT for up to 4 days, followed by concentration in vacuo for 4 h for any remaining solutions.

The crystal-form screen produced only one crystalline form, Form A. Form A crystalline solid was produced in a large number of solvents, particularly under thermocycling and cooling conditions. For example, Form A crystalline solid was produced under thermocycling and cooling conditions in acetonitrile, 2-butanone, ethyl acetate, and ethanol, among others. Amorphous (R)-oxybutynin D-malate was produced in certain solvents under evaporation conditions. For example, amorphous (R)-oxybutynin D-malate was produced under evaporation conditions in water, methanol, and dichloromethane, among others.

### Example 5: Production from Racemic Oxybutynin HCl

Racemic oxybutynin HCl salt (100 g) was suspended in water (600 mL). The mixture was heated to 30° C. until dissolution was observed. Seed crystals of the crystalline oxybutynin free-base were added, and the mixture was held at 30° C. Aqueous sodium hydroxide (1.0 eq of 1 M solution, 254 mL) was added dropwise over 4 hours to prevent formation of a gum. During base addition, a free-flowing white slurry was observed which became thicker over time. The reactor temperature was set to 20° C., and the mixture stirred overnight for 19 hours. The solids were isolated by filtration, and then dried at 40° C. under vacuum with nitrogen bleed for 20 hours. The overall yield of racemic oxybutynin (free base) was 95% (86.1 g) with an adjusted yield of 93% after subtracting seed crystals. The solids were a white powder, determined to be crystalline racemic oxybutynin free base.

Racemic oxybutynin free base (86.1 g) was combined with 2-propanol (400 mL). The mixture was heated to 50° C., resulting in a solution. Seed crystals of the D-malate salt of R-oxybutynin were added (0.55 g), followed by solid D-malic acid (24.2 g) with a rinse of 30 mL of 2-propanol to produce R-oxybutynin D-malate. The very thin slurry was maintained at 50 ºC for 1 hour, then cooled at 0.1° C./min to 20° C. and held at 20° C. for about 60 hours. An aliquot was taken to estimate yield (˜30%) and chiral purity (˜93% R, 86% ee) of R-oxybutynin D-malate. To increase yield, the mixture was slowly cooled at 0.1° C./min to 5° C. and held at 5° C. for 16 hours. A second aliquot indicated a slightly increased yield with comparable chiral purity (˜90% R, 80% ee). The mixture was filtered. The combined solids were washed with additional MTBE and air-dried for 1.5 hours. The yield was 41% (49.1 g).

R-oxybutynin D-malate salt (44 g) was combined with MIBK (methyl isobutyl ketone) (220 mL). The mixture was heated to 40° C. for 2 hours, cooled at 0.1° C./min to 5° C., and held at 5 ºC for about 12 hours. An aliquot of the re-crystallized product indicated 97% R, 3% S (94% ee) with the filtrate indicating a higher amount of the undesired isomer (27% R, 73% S). The product was isolated by vacuum filtration and air-dried for 1 hour. The wet cake product was still very wet (14% loss of MIBK up to 50° C. by TGA). The product was dried in a vacuum oven at 40° ° C. with nitrogen bleed overnight. The yield of recrystallized product was 93% (40.8 g). Use of MTBE in place of MIBK is also contemplated. The crystalline product was analyzed by XRPD and identified as Form A.

Thus, it was determined that by use of the chiral acid, D-malic acid, racemic oxybutynin may be converted to a crystalline (R)-oxybutynin salt. Eleven other chiral acids were tested for production of (R)-oxybutynin salt from racemic oxybutynin: L-tartaric acid, D-tartaric acid, L-(+)-lactic acid, D-glucuronic acid, D-gluconic acid, L-malic acid, (1R,3S)-(+)-camphoric acid, (S)-(+)-mandelic acid, (1R)-(−)-10-camphorsulfonic acid, L-pyroglutamic acid, and D-(−)-quinic acid. None of the other eleven chiral acids were successful in chiral resolution to produce an (R)-oxybutynin salt from racemic oxybutynin.

### Example 6: Production and Characterization of (R)-Oxybutynin L-Tartrate

Approximately 20 mg of (R)-oxybutynin free base was combined with 1.0 eq of L-tartaric acid. THF/MTBE solvent was added with isooctane added as antisolvent. Three crystallization techniques were used (slurry ripening, cooling, evaporation) and a temperature range of 5-40° C. to attempt to produce a crystalline form. Following evaporation, the experiment produced gum/unreacted chiral acid. To the gum/unreacted chiral acid, seed crystals of (R)-oxybutynin D-malate salt were added. This resulted in production of crystalline (R)-oxybutynin L-tartrate salt.

The (R)-oxybutynin L-tartrate salt was crystalline by XRPD and had a mix of birefringent plates and irregularly shaped particles by polarized light microscopy. FIG. 6 is the XRPD pattern for the (R)-oxybutynin L-tartrate. FIG. 7 is a FT-Raman spectrum for the (R)-oxybutynin L-tartrate. DSC analysis showed a large endotherm at 92° C. (ΔH=54 J/g). TGA analysis showed small weight loss of 1.4% from 50-100° C., possibly trapped solvent. FIG. 8 shows the DSC and TGA traces for the (R)-oxybutynin L-tartrate. Proton NMR confirmed a mono-salt (actual ration of 1.0:1 counterion: API ratio) with trace MTBE.

### Example 7: Chiral Salt Screen Results with Seed Crystals

Seed crystals of (R)-oxybutynin D-malate or (R)-oxybutynin L-tartrate were introduced to (R)-oxybutynin with various chiral acids in various solvents as listed in Table 3. The results are shown in Table 3. A=amorphous. S=isolated solid consistent with acid. C=birefringent (crystalline) salt hit.

TABLE 3

| | | | | | | Ethyl | | |
|---|---|---|---|---|---|---|---|---|
| Chiral Acid | | | | | | acetate/ | | 2- |
| (1.0 eq) | Isooctane | Heptane | Toluene | THF | Acetone | MTBE | Acetonitrile | propanol |
| L-tartaric acid | A | A | A | A | A | A | A | A |
| D-tartaric acid | A | A | A | A | A | A | A | A |
| L-(+)-lactic acid | A | A | A | A | A | A | A | A |
| D-glucuronic | A | A | A | A | A | A | A | A |
| acid | | | | | | | | |
| D-gluconic acid | A | A | A | S | S | A | A | A |
| L-malic acid | A | A | A | A | A | A | A | A |
| D-malic acid | A | A | A | A | A | A | A | C |
| (1R,3S)-(+)- | A | A | A | A | A | A | A | A |
| camphoric acid | | | | | | | | |
| (S)-(+)- | A | A | A | A | A | A | A | A |
| mandelic acid | | | | | | | | |
| (1R)-(−)-10- | A | A | A | A | A | A | A | A |
| camphorsulfonic | | | | | | | | |
| acid | | | | | | | | |
| L-pyroglutamic | A | A | A | A | A | A | A | A |
| acid | | | | | | | | |
| Quinic acid | A | A | A | S | S | S | S | S |

Further embodiments of the present invention:
Embodiment E1. A crystalline D-malate salt of (R)-oxybutynin.
Embodiment E2. The crystalline D-malate salt of Embodiment E1, which is the Form A crystalline salt.
Embodiment E3. The crystalline D-malate salt of Embodiment E2, characterized by an X-ray powder diffraction (XRPD) pattern comprising at least three peaks selected from the group consisting of peaks at 11.0, 14.3, 17.5, 19.8 and 22.0° 2θ+0.2° 2θ.
Embodiment E4. The crystalline D-malate salt of Embodiment E3, characterized by an XRPD pattern comprising peaks at 11.0, 17.5 and 22.0° 2θ+0.2° 2θ.
Embodiment E5. The crystalline D-malate salt of Embodiment E4, characterized by an XRPD pattern comprising peaks at 11.0, 14.3, 17.5, 19.8 and 22.0° 2θ+0.2° 2θ.
Embodiment E6. The crystalline D-malate salt of Embodiment E2, characterized by an XRPD pattern comprising at least five peaks selected from the group consisting of peaks at 11.0, 14.3, 17.5, 18.8, 19.8, 21.4, 22.0, 23.3, 24.1 and 33.2° 2θ+0.2° 2θ.
Embodiment E7. The crystalline D-malate salt of Embodiment E6, characterized by an XRPD pattern comprising peaks at 11.0, 14.3, 17.5, 18.8, 19.8, 21.4, 22.0, 23.3, 24.1 and 33.2° 2θ+0.2° 2θ.
Embodiment E8. The crystalline D-malate salt of Embodiment E2, having an XRPD pattern substantially as shown in FIG. 1.
Embodiment E9. The crystalline D-malate salt of any one of Embodiments E1-E8, having a differential scanning calorimetry (DSC) thermogram comprising a melting onset at about 108.1° C. and an endothermic peak at about 109.4° C.
Embodiment E10. The crystalline D-malate salt of Embodiment E9, having a differential scanning calorimetry (DSC) thermogram substantially as shown in FIG. 3.
Embodiment E11. A pharmaceutical composition comprising the crystalline D-malate salt of any one of Embodiments E1-E10 and one or more pharmaceutically acceptable excipients.
Embodiment E12. A method of treating a condition associated with pharyngeal airway collapse comprising administering to a subject in need thereof the crystalline D-malate salt of any one of Embodiments E1-E10 or a pharmaceutical composition of Embodiment E11.
Embodiment E13. The method of Embodiment E13, wherein the condition associated with pharyngeal airway collapse is sleep apnea or snoring.
Embodiment E14. The method of Embodiment E13, wherein the condition associated with pharyngeal airway collapse is obstructive sleep apnea (OSA).
Embodiment E15. An amorphous D-malate salt of (R)-oxybutynin.
Embodiment E16. A method of making crystalline (R)-oxybutynin D-malate of Form A, the method comprising adding D-malic acid to racemic oxybutynin in the presence of a solvent to produce (R)-oxybutynin D-malate of Form A.
Embodiment E17. The method of Embodiment E16, wherein the solvent is 2-propanol.
Embodiment E18. A method of making crystalline (R)-oxybutynin D-malate Form A, the method comprising adding D-malic acid to (R)-oxybutynin free base in the presence of ethyl acetate and MTBE.
Embodiment E19. The method of claim Embodiment E18, wherein the ethyl acetate and MTBE are in a volume ratio of about 1:4 of ethyl acetate:MTBE.
Embodiment E20. A crystalline L-tartrate salt of (R)-oxybutynin.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A crystalline D-malate salt of (R)-oxybutynin.

2. The crystalline D-malate salt of claim 1, which is the Form A crystalline salt.

3. The crystalline D-malate salt of claim 2, characterized by an X-ray powder diffraction (XRPD) pattern comprising at least three peaks selected from the group consisting of peaks at 11.0, 14.3, 17.5, 19.8 and 22.0° 2θ+0.2° 2θ.

4. The crystalline D-malate salt of claim 3, characterized by an XRPD pattern comprising peaks at 11.0, 17.5 and 22.0° 2θ+0.2° 2θ.

5. The crystalline D-malate salt of claim 4, characterized by an XRPD pattern comprising peaks at 11.0, 14.3, 17.5, 19.8 and 22.0° 2θ+0.2° 2θ.

6. The crystalline D-malate salt of claim 2, characterized by an XRPD pattern comprising at least five peaks selected from the group consisting of peaks at 11.0, 14.3, 17.5, 18.8, 19.8, 21.4, 22.0, 23.3, 24.1 and 33.2° 2θ+0.2° 2θ.

7. The crystalline D-malate salt of claim 6, characterized by an XRPD pattern comprising peaks at 11.0, 14.3, 17.5, 18.8, 19.8, 21.4, 22.0, 23.3, 24.1 and 33.2° 2θ+0.2° 2θ.

8. The crystalline D-malate salt of claim 2, having an XRPD pattern substantially as shown in FIG. 1.

9. The crystalline D-malate salt of claim 3, having a differential scanning calorimetry (DSC) thermogram comprising a melting onset at about 108.1° C. and an endothermic peak at about 109.4° C.

10. The crystalline D-malate salt of claim 9, having a differential scanning calorimetry (DSC) thermogram substantially as shown in FIG. 3.

11. A pharmaceutical composition comprising the crystalline D-malate salt of claim 3 and one or more pharmaceutically acceptable excipients.

12. A method of treating a condition associated with pharyngeal airway collapse comprising administering to a subject in need thereof the pharmaceutical composition of claim 11.

13. The method of claim 12, wherein the condition associated with pharyngeal airway collapse is sleep apnea or snoring.

14. The method of claim 13, wherein the condition associated with pharyngeal airway collapse is obstructive sleep apnea (OSA).

15. An amorphous D-malate salt of (R)-oxybutynin.

16. A method of making crystalline (R)-oxybutynin D-malate of Form A, the method comprising adding D-malic acid to racemic oxybutynin in the presence of a solvent to produce (R)-oxybutynin D-malate of Form A.

17. The method of claim 16, wherein the solvent is 2-propanol.

18. A method of making crystalline (R)-oxybutynin D-malate Form A, the method comprising adding D-malic acid to (R)-oxybutynin free base in the presence of ethyl acetate and MTBE.

19. The method of claim 18, wherein the ethyl acetate and MTBE are in a volume ratio of about 1:4 of ethyl acetate:MTBE.

20. A crystalline L-tartrate salt of (R)-oxybutynin.
